(19) United States
(12) Patent Application Publication
(10) Pub. No.: US 2026/0131157 A1
(43) Pub. Date: May 14, 2026

(54) METHODS OF USING ALTERNATING ELECTRIC FIELDS

(71) Applicant: NOVOCURE GMBH, Baar (CH)

(72) Inventor: Ori Farber, Haifa (IL)

(21) Appl. No.: 19/381,615

(22) Filed: Nov. 6, 2025

Related U.S. Application Data
(60) Provisional application No. 63/718,420, filed on Nov. 8, 2024.

(51) Int. Cl. A61N 1/40; A61K 31/495; A61N 5/10; A61P 35/00; C12Q 1/6886; G01N 33/574

(52) U.S. Cl. CPC A61N 1/40; A61K 31/495; A61N 5/10; A61P 35/00; C12Q 1/6886; G01N 33/5758; C12Q 2600/154; C12Q 2600/156; G01N 2333/904; G01N 2474/20

(57) ABSTRACT

Disclosed are methods of treating of a subject having cancer comprising applying an alternating electric field to a target site of the subject for a period of time, wherein the target site comprises one or more cancer cells, wherein the subject has an isocitrate dehydrogenase mutation (IDHmut). Disclosed are methods of treating a subject comprising identifying a subject with an IDHmut; and applying an alternating electric field to a target site of the subject for a period of time, wherein the target site comprises one or more cancer cells.

### CROSS-REFERENCE TO RELATED APPLICATIONS

This application claims the benefit of U.S. Provisional Ser. No. 63/718,420, filed on Nov. 8, 2024, which is incorporated by reference herein in its entirety.

### BACKGROUND

Patients diagnosed with glioblastoma (GBM) often have a poor prognosis and are usually treated with chemotherapy regimens associated with systemic toxicities. Tumor Treating Fields (TTFields) is a noninvasive, loco-regional treatment that disrupts cancer cell viability through a multimodal mechanism that includes antimitotic and antitumor immune effects.

Determining if specific patient subpopulations respond better to TTFields can be a promising use of TTFields in specific patients.

### BRIEF SUMMARY

Disclosed are methods of treating of a subject having cancer comprising applying an alternating electric field to a target site of the subject for a period of time, wherein the target site comprises one or more cancer cells, wherein the subject has an isocitrate dehydrogenase mutation (IDHmut).

Disclosed are methods of treating a subject comprising identifying a subject with an isocitrate dehydrogenase mutation (IDHmut); and applying an alternating electric field to a target site of the subject for a period of time, wherein the target site comprises one or more cancer cells.

Disclosed are methods of treating a subject comprising identifying a subject with methylguanine-methyltransferase-promoter (MGMT) methylation; and applying an alternating electric field to a target site of the subject for a period of time, wherein the target site comprises one or more cancer cells.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows an example of the NovoTTF-200A system for use in GBM.

FIG. 2 shows a Kaplan-Meier survival plot of OS for IDH-mutant patients.

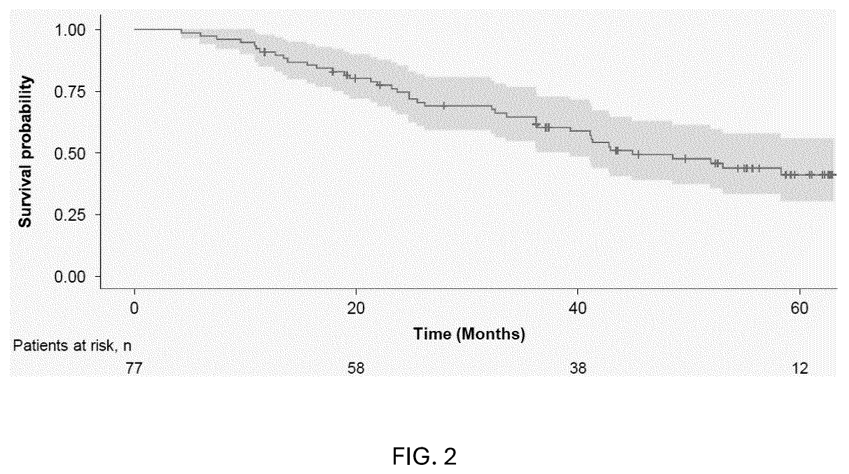

FIG. 3 shows a Kaplan-Meier survival plot of OS for IDH-mutant patients by TTFields usage level.

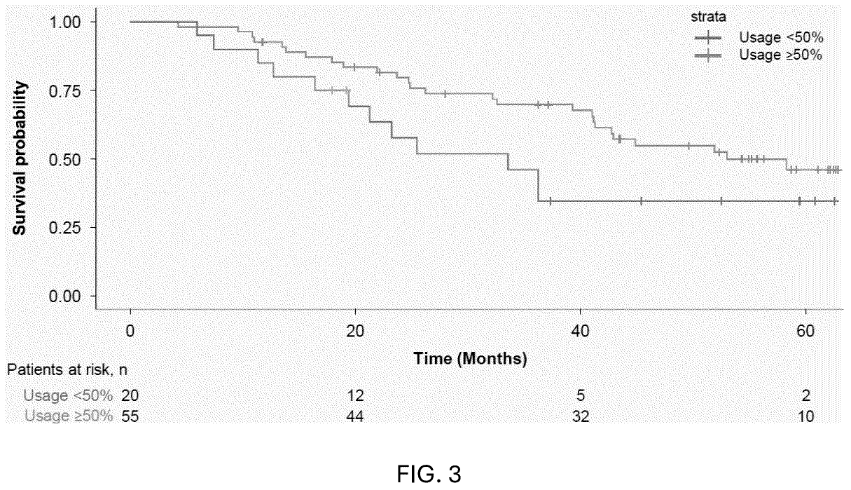

FIG. 4 shows a table with baseline characteristics in patients with IDHmut.

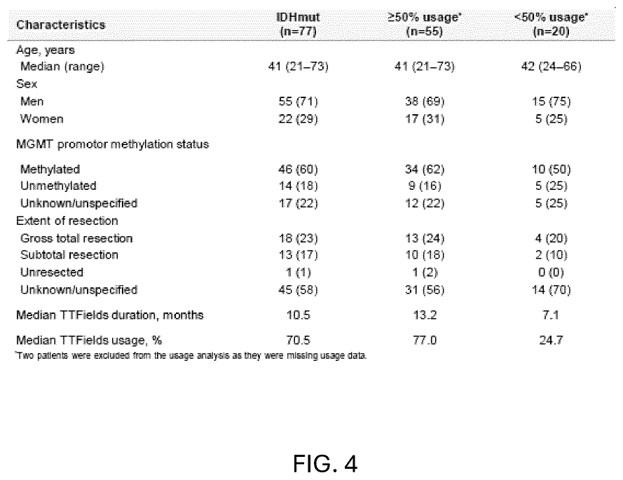

FIG. 5 shows a table showing overall survival of IDH-mutant patients.

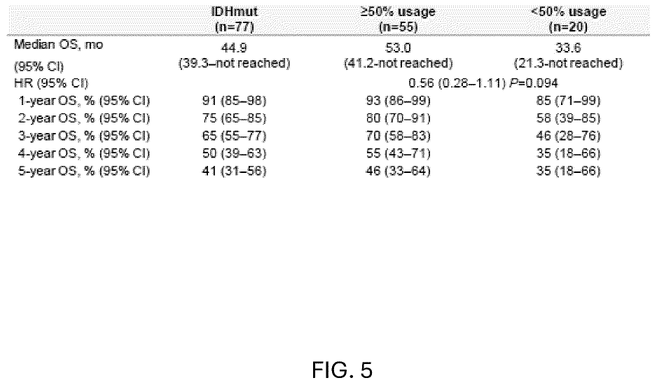

FIG. 6 shows a table with patient and treatment characteristics by IDHmut status.

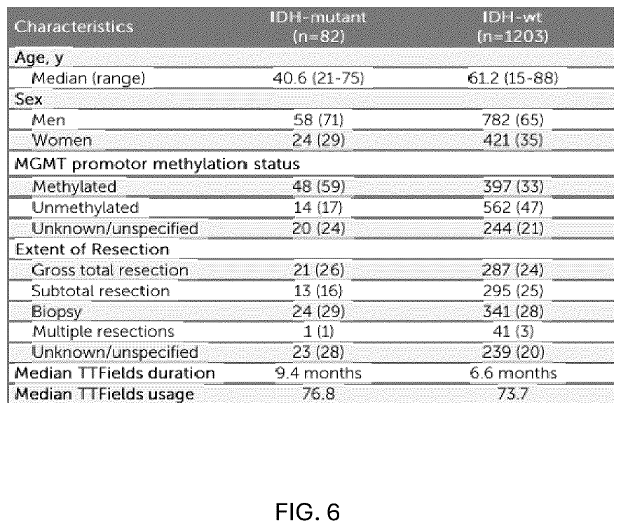

FIG. 7 shows a Kaplan-Meier survival plot by IDHmut status.

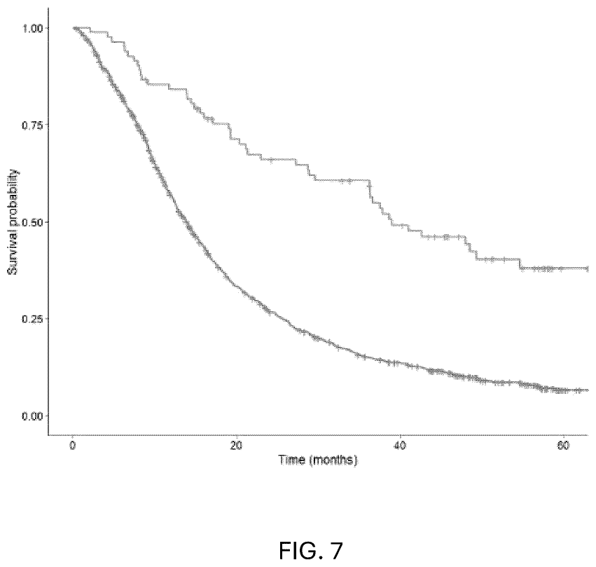

FIG. 8 shows overall survival of IDH-mutant patients.

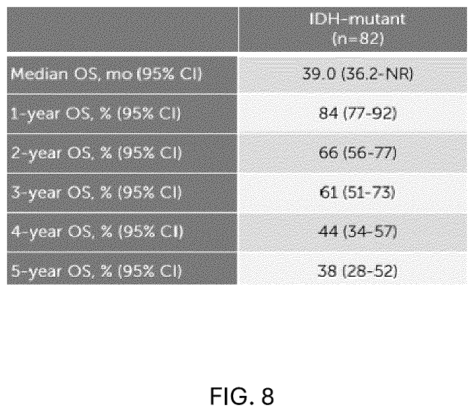

### DETAILED DESCRIPTION

The disclosed methods and compositions may be understood more readily by reference to the following detailed description of particular embodiments and the Example included therein and to the Figures and their previous and following description.

It is to be understood that the disclosed methods and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed methods and compositions are not limited to the particular methodology, protocols, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a” , “an”, and “the” include plural reference unless the context clearly dictates otherwise.

As used herein, a “target site” is a specific site or location within or present on a subject or patient. For example, a “target site” can refer to, but is not limited to a cell (e.g., a cancer cell), population of cells, organ, tissue, or a tumor. Thus, the phrase “target cell” can be used to refer to target site, wherein the target site is a cell. In some aspects, a “target cell” can be a cancer cell. In some aspects, organs that can be target sites include, but are not limited to, the lungs. In some aspects, a cell or population of cells that can be a target site or a target cell include, but are not limited to, a cancer cell (e.g., a lung cancer cell). In some aspects, a “target site” can be a tumor target site.

As used herein, a “target site” is a specific site or location within or present on a subject or patient. For example, a “target site” can refer to, but is not limited to a cell, population of cells, organ, tissue, tumor, or cancer cell. In some aspects, organs include, but are not limited to, lung, brain, pancreas, abdominal organs (e.g. stomach, intestine), ovary, breast, uterus, prostate, bladder, liver, colon, or kidney. In some aspects, a cell or population of cells include, but are not limited to, lung cells, brain cells, pancreatic cells, abdominal cells, ovarian cells, liver cells, colon cells, or kidney cells. In some aspects, a “target site” can be a tumor target site.

A “tumor target site” is a site or location within or present on a subject or patient that comprises or is adjacent to one or more cancer cells, previously comprised one or more tumor cells, or is suspected of comprising one or more tumor cells. For example, a tumor target site can refer to a site or location within or present on a subject or patient that is prone to metastases. Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g. a target site). In some aspects, the alternating electrical field can be in a single direction or multiple directional. In some aspects, alternating electric fields can be delivered through two pairs of transducer arrays that generate perpendicular fields within the treated target region. For example, for the Optune™ system (an alternating electric fields delivery system) one pair of electrodes is located to the left and right (LR) of the target region, and the other pair of electrodes is located anterior and posterior (AP) to the target region. Cycling the field between these two directions (i.e., LR and AP) ensures that a maximal range of cell orientations is targeted. In some aspects, an alternating electric field can be referred to as Tumor Treating Field (TTField).

As used herein, an “alternating electric field” applied to a tumor target site can be referred to as a “tumor treating field” or “TTField.” TTFields have been established as an anti-mitotic cancer treatment modality because they interfere with proper micro-tubule assembly during metaphase and eventually destroy the cells during telophase, cytokinesis, or subsequent interphase. TTFields target solid tumors and are described in U.S. Pat. No. 7,565,205, which is incorporated herein by reference in its entirety for its teaching of TTFields.

In-vivo and in-vitro studies show that the efficacy of alternating electric fields therapy increases as the intensity of the electrical field increases. Therefore, optimizing array placement on the area of a patient's tumor to increase the intensity in the desired region of the tumor can be performed with the Optune system. Array placement optimization may be performed by “rule of thumb” (e.g., placing the arrays on the tumor as close to the desired region of the target site (e.g., cancer cells) as possible), measurements describing the geometry of the patient's tumor, tumor dimensions. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target region as a function of the positions of the array and, in some aspects, take account for variations in the electrical property distributions within the same target region of different patients.

The term “subject” refers to the target of administration, e.g. an animal. Thus, the subject of the disclosed methods can be a vertebrate, such as a mammal. For example, the subject can be a human. The term does not denote a particular age or sex. Subject can be used interchangeably with “individual” or “patient.”

By “treat” is meant to administer or apply a therapeutic, such as alternating electric fields and/or a chemotherapy, to a subject, such as a human or other mammal (for example, an animal model), that has cancer or has an increased susceptibility for developing cancer, in order to prevent or delay a worsening of the effects of the cancer, or to partially or fully reverse the effects of the cancer (glioblastoma, ovarian, or lung metastatic carcinoma).

By “prevent” is meant to minimize the chance that a subject who has an increased susceptibility for developing cancer will develop cancer.

As used herein, the terms “administering” and “administration” refer to any method of providing a therapeutic, such as a chemotherapy to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration so as to treat a subject. In some aspects, administering comprises exposing. Thus, in some aspects, exposing a cancer cell to alternating electrical fields means administering alternating electrical fields to the cancer cell.

As used herein, the term “therapeutically effective amount” means an amount of a therapeutic, prophylactic, and/or diagnostic agent that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

The word “or” as used herein means any one member of a particular list and also includes any combination of members of that list.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present methods and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise alternating electric fields. In some aspects, the alternating electric field used in the methods disclosed herein is a tumor-treating field (TTFields). In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 and 500 kHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 180 and 220 kHz, or between 210 and 400 kHz. In some aspects, the frequency of the alternating electric fields can be 50 kHz, 100 kHz, 200 kHz, 300 kHz, 400 kHz, 500 kHz, or any frequency between. In some aspects, the frequency of the alternating electric field is from about 200 kHz to about 400 kHz, from about 250 kHz to about 350 kHz, and may be around 300 kHz.

In some aspects, the alternating electric field may be induced by an applied voltage of at least 50 V RMS. In some aspects, the alternating electric field can be induced by an applied voltage of at least 25-200 V RMS. In some aspects, the alternating electric field can be induced by an applied voltage of at least about 5, 10, 15, 20, 25, 50, 75, 100, 125, 150, 175, 200, 225, 250, 275 or 300 V RMS.

In some aspects, the field strength of the alternating electric fields can be between 1 and 4 V/cm RMS. In some aspects, different field strengths can be used (e.g., between 0.1 and 10 V/cm). In some aspects, the field strength can be 1.75 V/cm RMS. In some embodiments the field strength is at least 1 V/cm. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two-hour duration.

In some aspects, the exposure may last for at least 6 hours, at least 12 hours, at least 24 hours, at least 36 hours, at least 48 hours, or at least 72 hours or more. In some aspects, the exposure can be consecutive or cumulative. In some aspects, the consecutive exposure may last for at least 6 hours, at least 12 hours, at least 24 hours, at least 36 hours, at least 48 hours, or at least 72 hours or more. In some aspects, the cumulative exposure may last for at least 42 hours, at least 84 hours, at least 168 hours, at least 250 hours, at least 400 hours, at least 500 hours, at least 750 hours, or more. In some aspects, there can be a break in treatment and the alternating electric fields are applied at least 50%, 60%, 70%, or 80% of treatment time. For example, in some aspects, cumulative exposure can be for at least 12 hours in a period of 24 hours.

The disclosed methods comprise applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell.

In some aspects, the exposure may last for at least 6 hours, at least 12 hours, at least 24 hours, at least 36 hours, at least 48 hours, or at least 72 hours or more.

In addition, when the alternating electric field is applied to a subject, the period of time that the alternating electric field is applied may be measured in terms of a continuous period of time or a cumulative period of time. That is, the period of time that the alternating electric field is applied may include a single session (i.e., continuous application) as well as multiple sessions with minor breaks in between sessions (i.e., consecutive applications for a cumulative period). For example, even within a continuous application, a subject is allowed to take breaks during treatment with an alternating electric field device and is only expected to have the device positioned on the body and operational for at least about 50%, at least about 60%, at least about 70%, or at least about 80% of the total treatment period (e.g., over a course of one day, one week, two weeks, one month, two months, three months, four months, five months, etc.). For example, the alternating electric field can be applied for at least 12 hours, 16 hours, or 18 hours cumulative each day for a week, a month, two months, three months, etc.

### C. Methods of Treatment

### 1. Treating a Subject Having an Idhmut

Isocitrate dehydrogenase (IDH) (EC 1.1.1.42) and (EC 1.1.1.41) is an enzyme that catalyzes the oxidative decarboxylation of isocitrate, producing alpha-ketoglutarate (α-ketoglutarate) and CO2. In humans, IDH exists in three isoforms: IDH3 catalyzes the third step of the citric acid cycle while converting NAD+to NADH in the mitochondria. The isoforms IDH1 and IDH2 catalyze the same reaction outside the context of the citric acid cycle and use NADP+ as a cofactor instead of NAD+. They localize to the cytosol as well as the mitochondrion and peroxisome.

Disclosed are methods of treating of a subject having cancer comprising applying an alternating electric field to a target site of the subject for a period of time, wherein the target site comprises one or more cancer cells, wherein the subject has an isocitrate dehydrogenase mutation (IDHmut). In some aspects, the IDHmut can be a mutation in IDH1 or IDH2.

In some aspects, a subject having an IDHmut can have any one of many known IDHmuts. In some aspects, the IDHmut can be a DNA point mutation in IDH1 or IDH2 that changes the amino-acid sequence of the enzyme (i.e., a protein-coding missense mutation). For example, IDH1 (cytosolic/peroxisomal enzyme) mutations can be, but are not limited to, R132H (CGT→CAT) in exon 4, R132C (CGT→TGT), R132S (CGT→AGT), R132G (CGT→GGT), R132L (CGT→CTT), R132P, or R132V. In some aspects, IDH2 (mitochondrial enzyme) mutations can be, but are not limited to, R172K (AGG→AAG), R172M, R172S, R172G, R172W, R172T, or R140Q.

In some aspects, the methods further comprise administering a therapeutically effective amount of chemotherapy to the subject. Thus, disclosed are methods of treating of a subject having cancer comprising applying an alternating electric field to a target site of the subject for a period of time; and administering a therapeutically effective amount of a chemotherapy to the subject, wherein the target site comprises one or more cancer cells, wherein the subject has an IDHmut. In some aspects, the chemotherapy can be any known chemotherapy. In some aspects, the chemotherapy can be temozolomide (TMZ). In some aspects, the TMZ is administered at 50-100 mg/m² per dose. In some aspects, the TMZ is administered at 75 mg/m² per dose. In some aspects, the TMZ used in the methods disclosed herein is administered daily at 75 mg/m² per dose for 6 weeks (e.g., the 6 weeks concomitant with radiation therapy) and/or at 150-200 mg/m² for five days every 28 days. In some aspects, the chemotherapy is administered before, during, and/or after applying the alternating electric field.

In some aspects, the methods further comprise administering a therapeutically effective amount of radiation therapy to the subject. Thus, disclosed are methods of treating of a subject having cancer comprising applying an alternating electric field to a target site of the subject for a period of time; administering a therapeutically effective amount of a chemotherapy to the subject, and administering a therapeutically effective amount of a radiation therapy to the subject, wherein the target site comprises one or more cancer cells, wherein the subject has an IDHmut. In some aspects, the total dose of radiation therapy is about 60 Gy. Thus, in some aspects, radiation therapy is administered at 2 Gy/day for 5 days/week for 6 weeks. In some aspects, radiation therapy can be administered to the subject for 4 to 8 weeks.

In some aspects, the chemotherapy is administered before, during, and/or after applying the alternating electric field. In some aspects, the chemotherapy is administered before, during, and/or after radiation therapy. In some aspects, the radiation therapy is administered before, during, and/or after applying the alternating electric field. In some aspects, the radiation therapy is administered before, during, and/or after administering the chemotherapy. In some aspects, applying the alternating electric fields and administering a chemotherapy simultaneously can mean applying the alternating electric fields within 5, 10, 15, 20, 25, 30, 35, 40, 45, 50, 55, or 60 minutes before or after administering the chemotherapy. In some aspects, applying the alternating electric fields and administering radiation therapy simultaneously can mean applying the alternating electric fields within 5, 10, 15, 20, 25, 30, 35, 40, 45, 50, 55, or 60 minutes before or after administering the radiation therapy. In some aspects, the alternating electric fields can be applied and the chemotherapy and/or radiation therapy administered at least 1, 2, 3, 4, 5, 6, 7, 8, 9, 10, 11, 12, 13, 14, 15, 16, 17, 18, 19, 20, 21, 22, 23, or 24 hours from each other.

In some aspects of the disclosed methods of treating, the subject has not previously undergone radiotherapy. In some aspects, the subject has not previously undergone surgery or biopsy. In some aspects, the subject has not previously undergone chemotherapy.

In some aspects of the disclosed methods of treating, the subject has previously undergone a prior treatment. For instance, in some aspects, the subject has previously undergone radiotherapy. In some aspects, the subject has previously undergone surgery (e.g., debulking surgery) or biopsy. In some aspects, the subject has previously undergone chemotherapy. In some aspects, the subject has previously undergone one or more of surgery, chemotherapy, and/or radiotherapy. In some aspects, the subject has progressed after or during a cancer treatment, such as, but not limited to, surgery, chemotherapy, or radiotherapy. In some aspects, the subject has not progressed after surgery or biopsy, chemotherapy, and/or radiotherapy.

In some aspects, the subject has previously undergone chemotherapy. In some aspects, the subject is currently undergoing chemotherapy. In some aspects, the subject has previously failed chemotherapy.

In some aspects of the disclosed methods of treating, the subject having cancer has brain cancer (e.g., astrocytoma, glioblastoma). In some aspects, the subject has IDHmut WHO grade 4 astrocytoma (astrocytoma IDHmut/G4).

In some aspects, the subject has methylguanine-methyltransferase (MGMT) promoter methylation.

In some aspects, the subject has ≥50% compliance with applying the alternating electric field. Thus, regardless of what the treatment regimen is for treating with the alternating electric field, the subject can be in ≥50% compliance of that treatment regimen. For example, if the subject is to receive the alternating electric field 24 hrs a day then, in some aspects of the disclosed methods, the subject receives the alternating electric field for ≥12 hrs a day.

In some aspects of the disclosed methods of treating, the target site of the subject comprises one or more cancer cells. In some aspects, the target site comprises one or more brain cancer cells such as astrocytoma cells or glioblastoma cells.

In some aspects, the subject has not previously been treated with an alternating electrical field. In some aspects, the subject is not currently being treated with any type of electric field other than an alternating electrical field.

In some aspects of the disclosed methods of treating, after treatment the subject has an increased overall survival. In some aspects, increased overall survival can be an increase of about 1, 2, 3, 4, 5, 6, 7, 8, 9, 10, 11, or 12 months. In some aspects, an increased overall survival is compared to subjects administered an alternating electric field alone, TMZ alone, or an alternating electric field plus TMZ alone.

In some aspects, after treatment the subject has an increased survival probability as compared to a subject without an IDHmut.

In some aspects, after treatment the subject has an increased overall survival as compared to a subject without an IDHmut. In some aspects, after treatment the subject has an increased overall survival when the subject has ≥50% compliance with applying the alternating electric field compared to a subject that has ≤50% compliance.

In some aspects, the frequency of the alternating electric fields is between 50 kHz and 1 MHz. In some aspects, the frequency of the alternating electric field is 150 or 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 0.9 V/cm RMS. In some aspects, the alternating electric field has a field strength of any of those described herein.

### 2. Identifying a Subject With Idhmut

Disclosed are methods of treating a subject comprising identifying a subject with an isocitrate dehydrogenase mutation (IDHmut); and applying an alternating electric field to a target site of the subject for a period of time, wherein the target site comprises one or more cancer cells.

In some aspects, identifying a subject with an IDHmut comprises identification at the protein level by immunohistochemistry (IHC) or at the DNA (or RNA) level by sequencing. Known techniques can be used to identify the IDHmut. For example, techniques described in Preusser et al. J Neuropathol Exp Neurol. 2011 Aug; 70(8):715-23; Satomi et al., Brain Tumor Pathology, 29 Jul. 2022, Volume 39, pages 210-217, (2022); or Choate et al. PLoS ONE 18(9): e0291666, each incorporated by reference herein, can be used.

In some aspects, the subject identified as having an IDHmut can have any one of many known IDHmuts. In some aspects, the IDHmut can be a DNA point mutation in IDH1 or IDH2 that changes the amino-acid sequence of the enzyme (i.e., a protein-coding missense mutation). For example, IDH1 (cytosolic/peroxisomal enzyme) mutations can be, but are not limited to, R132H (CGT→CAT) in exon 4, R132C (CGT→TGT), R132S (CGT→AGT), R132G (CGT→GGT), R132L (CGT→CTT), R132P, or R132V. In some aspects, IDH2 (mitochondrial enzyme) mutations can be, but are not limited to, R172K (AGG→AAG), R172M, R172S, R172G, R172W, R172T, or R140Q.

In some aspects, the methods further comprise administering a therapeutically effective amount of chemotherapy to the subject. Thus, disclosed are methods of treating of a subject having cancer comprising applying an alternating electric field to a target site of the subject for a period of time; and administering a therapeutically effective amount of a chemotherapy to the subject, wherein the target site comprises one or more cancer cells, wherein the subject has an IDHmut. In some aspects, the chemotherapy can be any known chemotherapy. In some aspects, the chemotherapy can be temozolomide (TMZ). In some aspects, the TMZ is administered at 50-100 mg/m² per dose. In some aspects, the TMZ is administered at 75 mg/m² per dose. In some aspects, the TMZ used in the methods disclosed herein is administered daily at 75 mg/m² per dose for 6 weeks (e.g., the 6 weeks concomitant with radiation therapy) and/or at 150-200 mg/m² for five days every 28 days. In some aspects, the chemotherapy is administered before, during, and/or after applying the alternating electric field.

In some aspects, the methods further comprise administering a therapeutically effective amount of radiation therapy to the subject. Thus, disclosed are methods of treating of a subject having cancer comprising applying an alternating electric field to a target site of the subject for a period of time; administering a therapeutically effective amount of a chemotherapy to the subject, and administering a therapeutically effective amount of a radiation therapy to the subject, wherein the target site comprises one or more cancer cells, wherein the subject has an IDHmut. In some aspects, the total dose of radiation therapy is about 60 Gy. Thus, in some aspects, radiation therapy is administered at 2 Gy/day for 5 days/week for 6 weeks. In some aspects, radiation therapy can be administered to the subject for 4 to 8 weeks.

In some aspects, the chemotherapy is administered before, during, and/or after applying the alternating electric field. In some aspects, the chemotherapy is administered before, during, and/or after radiation therapy. In some aspects, the radiation therapy is administered before, during, and/or after applying the alternating electric field. In some aspects, the radiation therapy is administered before, during, and/or after administering the chemotherapy. In some aspects, applying the alternating electric fields and administering a chemotherapy simultaneously can mean applying the alternating electric fields within 5, 10, 15, 20, 25, 30, 35, 40, 45, 50, 55, or 60 minutes before or after administering the chemotherapy. In some aspects, applying the alternating electric fields and administering radiation therapy simultaneously can mean applying the alternating electric fields within 5, 10, 15, 20, 25, 30, 35, 40, 45, 50, 55, or 60 minutes before or after administering the radiation therapy. In some aspects, the alternating electric fields can be applied and the chemotherapy and/or radiation therapy administered at least 1, 2, 3, 4, 5, 6, 7, 8, 9, 10, 11, 12, 13, 14, 15, 16, 17, 18, 19, 20, 21, 22, 23, or 24 hours from each other.

In some aspects of the disclosed methods of treating, the subject has not previously undergone radiotherapy. In some aspects, the subject has not previously undergone surgery or biopsy. In some aspects, the subject has not previously undergone chemotherapy.

In some aspects of the disclosed methods of treating, the subject has previously undergone a prior treatment. For instance, in some aspects, the subject has previously undergone radiotherapy. In some aspects, the subject has previously undergone surgery (e.g., debulking surgery) or biopsy. In some aspects, the subject has previously undergone chemotherapy. In some aspects, the subject has previously undergone one or more of surgery, chemotherapy, and/or radiotherapy. In some aspects, the subject has progressed after or during a cancer treatment, such as, but not limited to, surgery, chemotherapy, or radiotherapy. In some aspects, the subject has not progressed after surgery or biopsy, chemotherapy, and/or radiotherapy.

In some aspects, the subject has previously undergone chemotherapy. In some aspects, the subject is currently undergoing chemotherapy. In some aspects, the subject has previously failed chemotherapy.

In some aspects of the disclosed methods of treating, the subject having cancer has brain cancer (e.g., astrocytoma, glioblastoma). In some aspects, the subject has IDHmut WHO grade 4 astrocytoma (astrocytoma IDHmut/G4).

In some aspects, the subject has methylguanine-methyltransferase (MGMT) promoter methylation.

In some aspects, the subject has ≥50% compliance with applying the alternating electric field. Thus, regardless of what the treatment regimen is for treating with the alternating electric field, the subject can be in ≥50% compliance of that treatment regimen. For example, if the subject is to receive the alternating electric field 24 hrs a day then, in some aspects of the disclosed methods, the subject receives the alternating electric field for ≥12 hrs a day.

In some aspects, the target site comprises one or more brain cancer cells such as astrocytoma cells or glioblastoma cells.

In some aspects, the subject has not previously been treated with an alternating electrical field. In some aspects, the subject is not currently being treated with any type of electric field other than an alternating electrical field.

In some aspects of the disclosed methods of treating, after treatment the subject has an increased overall survival. In some aspects, increased overall survival can be an increase of about 1, 2, 3, 4, 5, 6, 7, 8, 9, 10, 11, or 12 months. In some aspects, an increased overall survival is compared to subjects administered an alternating electric field alone, TMZ alone, or an alternating electric field plus TMZ alone.

In some aspects, after treatment the subject has an increased survival probability as compared to a subject without an IDHmut.

In some aspects, after treatment the subject has an increased overall survival as compared to a subject without an IDHmut. In some aspects, after treatment the subject has an increased overall survival when the subject has ≥50% compliance with applying the alternating electric field compared to a subject that has ≤50% compliance.

In some aspects, the frequency of the alternating electric fields is between 50 kHz and 1 MHz. In some aspects, the frequency of the alternating electric field is 150 or 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 0.9 V/cm RMS. In some aspects, the alternating electric field has a field strength of any of those described herein.

### 3. Identifying a Subject with Methylguanine-methyltransferase-promoter (MGMT) Methylation

Disclosed are methods of treating a subject comprising identifying a subject with methylguanine-methyltransferase-promoter (MGMT) methylation; and applying an alternating electric field to a target site of the subject for a period of time, wherein the target site comprises one or more cancer cells.

In some aspects, the MGMT promoter methylation can be increased methylation compared to a subject not having cancer or not having an IDHmut.

In some aspects, identifying a subject with MGMT methylation comprises performing quantitative MS-PCR, pyrosequencing, and/or any other known method of determining MGMT methylation.

In some aspects, the subject has an IDHmut. In some aspects, the subject having an IDHmut can have any one of many known IDHmuts. In some aspects, the IDHmut can be a DNA point mutation in IDH1 or IDH2 that changes the amino-acid sequence of the enzyme (i.e., a protein-coding missense mutation). For example, IDH1 (cytosolic/peroxisomal enzyme) mutations can be, but are not limited to, R132H (CGT→CAT) in exon 4, R132C (CGT→TGT), R132S (CGT→AGT), R132G (CGT→GGT), R132L (CGT→CTT), R132P, or R132V. In some aspects, IDH2 (mitochondrial enzyme) mutations can be, but are not limited to, R172K (AGG→AAG), R172M, R172S, R172G, R172W, R172T, or R140Q.

In some aspects, the methods further comprise administering a therapeutically effective amount of chemotherapy to the subject. Thus, disclosed are methods of treating of a subject having cancer comprising applying an alternating electric field to a target site of the subject for a period of time; and administering a therapeutically effective amount of a chemotherapy to the subject, wherein the target site comprises one or more cancer cells, wherein the subject has an IDHmut. In some aspects, the chemotherapy can be any known chemotherapy. In some aspects, the chemotherapy can be temozolomide (TMZ). In some aspects, the TMZ is administered at 50-100 mg/m² per dose. In some aspects, the TMZ is administered at 75 mg/m² per dose. In some aspects, the TMZ used in the methods disclosed herein is administered daily at 75 mg/m² per dose for 6 weeks (e.g., the 6 weeks concomitant with radiation therapy) and/or at 150-200 mg/m² for five days every 28 days. In some aspects, the chemotherapy is administered before, during, and/or after applying the alternating electric field.

In some aspects, the methods further comprise administering a therapeutically effective amount of radiation therapy to the subject. Thus, disclosed are methods of treating of a subject having cancer comprising applying an alternating electric field to a target site of the subject for a period of time; administering a therapeutically effective amount of a chemotherapy to the subject, and administering a therapeutically effective amount of a radiation therapy to the subject, wherein the target site comprises one or more cancer cells, wherein the subject has an IDHmut. In some aspects, the total dose of radiation therapy is about 60 Gy. Thus, in some aspects, radiation therapy is administered at 2 Gy/day for 5 days/week for 6 weeks. In some aspects, radiation therapy can be administered to the subject for 4 to 8 weeks.

In some aspects, the chemotherapy is administered before, during, and/or after applying the alternating electric field. In some aspects, the chemotherapy is administered before, during, and/or after radiation therapy. In some aspects, the radiation therapy is administered before, during, and/or after applying the alternating electric field. In some aspects, the radiation therapy is administered before, during, and/or after administering the chemotherapy. In some aspects, applying the alternating electric fields and administering a chemotherapy simultaneously can mean applying the alternating electric fields within 5, 10, 15, 20, 25, 30, 35, 40, 45, 50, 55, or 60 minutes before or after administering the chemotherapy. In some aspects, applying the alternating electric fields and administering radiation therapy simultaneously can mean applying the alternating electric fields within 5, 10, 15, 20, 25, 30, 35, 40, 45, 50, 55, or 60 minutes before or after administering the radiation therapy. In some aspects, the alternating electric fields can be applied and the chemotherapy and/or radiation therapy administered at least 1, 2, 3, 4, 5, 6, 7, 8, 9, 10, 11, 12, 13, 14, 15, 16, 17, 18, 19, 20, 21, 22, 23, or 24 hours from each other.

In some aspects of the disclosed methods of treating, the subject has not previously undergone radiotherapy. In some aspects, the subject has not previously undergone surgery or biopsy. In some aspects, the subject has not previously undergone chemotherapy.

In some aspects of the disclosed methods of treating, the subject has previously undergone a prior treatment. For instance, in some aspects, the subject has previously undergone radiotherapy. In some aspects, the subject has previously undergone surgery (e.g., debulking surgery) or biopsy. In some aspects, the subject has previously undergone chemotherapy. In some aspects, the subject has previously undergone one or more of surgery, chemotherapy, and/or radiotherapy. In some aspects, the subject has progressed after or during a cancer treatment, such as, but not limited to, surgery, chemotherapy, or radiotherapy. In some aspects, the subject has not progressed after surgery or biopsy, chemotherapy, and/or radiotherapy.

In some aspects, the subject has previously undergone chemotherapy. In some aspects, the subject is currently undergoing chemotherapy. In some aspects, the subject has previously failed chemotherapy.

In some aspects of the disclosed methods of treating, the subject having cancer has brain cancer (e.g., astrocytoma, glioblastoma). In some aspects, the subject has IDHmut WHO grade 4 astrocytoma (astrocytoma IDHmut/G4).

In some aspects, the subject has ≥50% compliance with applying the alternating electric field. Thus, regardless of what the treatment regimen is for treating with the alternating electric field, the subject can be in ≥50% compliance of that treatment regimen. For example, if the subject is to receive the alternating electric field 24 hrs a day then, in some aspects of the disclosed methods, the subject receives the alternating electric field for ≥12 hrs a day.

In some aspects, the target site comprises one or more brain cancer cells such as astrocytoma cells or glioblastoma cells, non-small cell lung cancer cells, pancreatic cancer cells, hepatocellular cancer cells, brain cancer cells, breast cancer cells, colon cancer cells, ovarian cancer cells, mesothelioma cells, or renal cancer cells.

In some aspects, the subject has not previously been treated with an alternating electrical field. In some aspects, the subject is not currently being treated with any type of electric field other than an alternating electrical field.

In some aspects of the disclosed methods of treating, after treatment the subject has an increased overall survival. In some aspects, increased overall survival can be an increase of about 1, 2, 3, 4, 5, 6, 7, 8, 9, 10, 11, or 12 months. In some aspects, an increased overall survival is compared to subjects administered an alternating electric field alone, TMZ alone, or an alternating electric field plus TMZ alone.

In some aspects, after treatment the subject has an increased survival probability as compared to a subject without an IDHmut.

In some aspects, after treatment the subject has an increased overall survival as compared to a subject without an IDHmut. In some aspects, after treatment the subject has an increased overall survival when the subject has ≥50% compliance with applying the alternating electric field compared to a subject that has ≤50% compliance.

In some aspects, the frequency of the alternating electric fields is between 50 kHz and 1 MHz. In some aspects, the frequency of the alternating electric field is 150 or 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 0.9 V/cm RMS. In some aspects, the alternating electric field has a field strength of any of those described herein.

### D. Dosing Regimen

In some aspects, a single dosing regimen can be four weeks (e.g., 28 days). In some aspects, the alternating electric field is applied daily for the entire four weeks. In some aspects, the alternating electric field is applied daily for at least 2, 4, 6, 8, 10, 12, 14, 16, 18, 20, 22, or 24 hours/day. In some aspects, subjects that use the alternating electric field for ≥ the assigned dosing regimen are considered high usage subjects. In some aspects, subjects that use the alternating electric field for ≤ the assigned dosing regimen are considered low usage subjects.

In some aspects, a dosing regimen comprises administration of a chemotherapeutic, such as temozolomide (TMZ) while TTFields are applied continuously. Thus, in some aspects, the dosing regimen described herein can further comprise TMZ dosing that follows the EF-14 clinical trial (NCT00916409). In some aspects, the first dosing regimen (or cycle 1) of TMZ orally can occur four weeks after completing a TMZ+Radiotherapy phase. In some aspects, TMZ can be administered for 6-12 cycles of maintenance treatment. In some aspects, dosing regimen 1 is 150 mg/m 2 once daily for 5 days followed by 23 days without TMZ treatment (although alternating electric fields can be applied during this time).

In some aspects, at the start of dosing regimen (Cycle) 2, the dose is escalated to 200 mg/m2 , if the CTC non-hematologic toxicity for Cycle 1 is Grade ≤2 (except for alopecia, nausea and vomiting), absolute neutrophil count (ANC) is ≥1.5×109/L, and the platelet count is ≥100×109/L. In some aspect, the dose of TMZ remains at 200 mg/m 2 per day for the first 5 days of each subsequent cycle except if toxicity occurs in that case dose can be reduced to 100 mg/m2 . If the dose was not escalated at Cycle 2, escalation should not be done in subsequent cycles.

In some aspects, the dosing regimen comprises radiation therapy (radiotherapy). In some aspects, the radiation therapy is administered five days a week for six weeks. In some aspects, a daily dose of radiation is 2Gy. In some aspects, a total dose of radiation given to a subject during treatment is 60Gy.

### Examples

### A. Example: Outcomes of Patients with Isocitrate Dehydrogenase-Mutant WHO Grade 4 Astrocytoma Treated with Tumor Treating Fields (TTFields) Therapy: a Real-World Analysis

### 1. Introduction

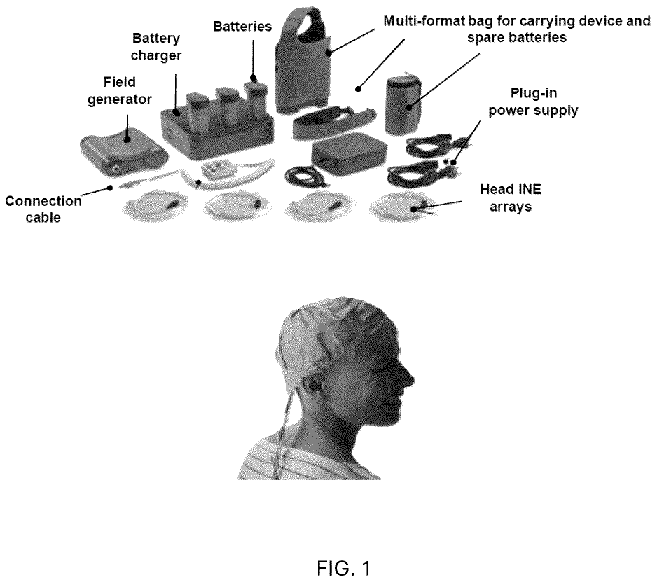

Patients diagnosed with glioblastoma (GBM) often have a poor prognosis and are usually treated with chemotherapy regimens associated with systemic toxicities. Tumor Treating Fields (TTFields) is a noninvasive, loco-regional treatment delivered via a portable medical device (FIG. 1) that disrupts cancer cell viability through a multimodal mechanism that includes antimitotic and antitumor immune effects.

TTFields therapy is FDA-approved and CE-marked for the treatment of GBM (WHO grade 4 glioma in Europe) based on results from the randomized, controlled, pivotal phase 3 EF-14 study (NCT00916409), which demonstrated improvement in progression-free survival and overall survival (OS).

TTFields therapy is also a “preferred” “category 1” recommendation for newly diagnosed GBM by the National Comprehensive Cancer Network guidelines. The EF-14 study included patients with the isocitrate dehydrogenase (IDH) mutation (IDHmut), (since reclassified as IDHmut WHO grade 4 astrocytoma [astrocytoma IDHmut/G4]) which confers a more favorable prognosis than IDH wild type.

However, large real-world datasets evaluating the impact of astrocytoma IDHmut/G4 on survival outcomes are lacking.

Disclosed are real-world outcomes of patients diagnosed with astrocytoma IDHmut/G4 treated with TTFields in the United States.

### 2. Methods

Electronic health records from the xCures real-world data platform were analyzed to identify US patients with newly diagnosed glioblastoma receiving TTFields, and confirmed IDHmut status.

Overall Survival (OS) was evaluated for the IDH-mutant cohort overall and by TTFields device usage, with patients that had ≥30 days' treatment categorized into high (≥50%) and low (<50%) usage group.

Device usage was quantified as the average percentage of time the device was operational during the initial 6 months of therapy.

Kaplan-Meier analysis and univariate Cox regression was used to assess survival.

### 3. Results

Of 1285 patients with known IDH status, 77 had an IDHmut; 55 were included in the high usage group and 20 were included in the low usage group.

Patients with high TTFields usage had increased OS compared with low TTFields usage (hazard ratio: 0.56[0.28-1.12], P=0.094).

Of the 22 patients with ≥4 years of survival, 5 were still on therapy at data cutoff.

### 4. Conclusions

Findings from an unselected patient population strongly suggest that patients with astrocytoma IDHmut/G4 benefit from TTFields therapy; this amplifies findings from the pivotal EF-14 study. The xCures platform demonstrates utility in systematically assessing real-world outcomes for a rare glioma subtype.

### Embodiments

Embodiment 1: A method of treating of a subject having cancer comprising: applying an alternating electric field to a target site of the subject for a period of time, wherein the target site comprises one or more cancer cells, wherein the subject has an isocitrate dehydrogenase mutation (IDHmut).

Embodiment 2: The method of embodiment 1, further comprising administering a therapeutically effective amount of chemotherapy to the subject.

Embodiment 3: The method of embodiment 2, wherein the chemotherapy is temozolomide (TMZ).

Embodiment 4: The method of any one of embodiments 2-3, wherein the chemotherapy is administered before, during, and/or after applying the alternating electric field.

Embodiment 5: The method of any of the preceding embodiments, wherein the subject has brain cancer.

Embodiment 6: The method of any of the preceding embodiments, wherein the subject has IDHmut WHO grade 4 astrocytoma (astrocytoma IDHmut/G4).

Embodiment 7: The method of any of the preceding embdiment s, wherein the subject has methylguanine-methyltransferase-promoter methylation.

Embodiment 8: The method of any of the preceding embodiments, wherein the subject has ≥50% compliance with applying the alternating electric field.

Embodiment 9: The method of any of the preceding embodiments, wherein after treatment the subject has an increased survival probability compared to subjects having wild type IDH.

Embodiment 10: The method of any of the preceding embodiments, wherein after treatment the subject has an increased overall survival compared to subjects having wild type IDH.

Embodiment 11: method of any of the preceding embodiments, wherein after treatment the subject has an increased overall survival when the subject has ≥50% compliance with applying the alternating electric field compared to a subjecting that has ≤50% compliance.

Embodiment 12: The method of any of the preceding embodiments, further comprising administering radiation therapy to the subject.

Embodiment 13: The method of embodiment 11, wherein the radiation therapy is administered before, during, and/or after applying the alternating electric field.

Embodiment 14: The method of embodiment 11 or 12, wherein the radiation therapy is administered before, during, and/or after administering the chemotherapy.

Embodiment 15: A method of treating a subject comprising: identifying a subject with an isocitrate dehydrogenase mutation (IDHmut); and applying an alternating electric field to a target site of the subject for a period of time, wherein the target site comprises one or more cancer cells.

Embodiment 16: The method of embodiment 15, wherein identifying a subject with an IDHmut comprises immunohistochemistry and/or sequencing.

Embodiment 17: The method of any one of embodiments 15-16, further comprising administering a therapeutically effective amount of chemotherapy to the subject.

Embodiment 18: The method of embodiment 17, wherein the chemotherapy is temozolomide (TMZ).

Embodiment 19: The method of any one of embodiments 17-18, wherein the chemotherapy is administered before, during, and/or after applying the alternating electric field.

Embodiment 20: The method of any of embodiments 15-19, wherein the subject has brain cancer.

Embodiment 21: The method of any of embodiments 15-20, wherein the subject has IDHmut WHO grade 4 astrocytoma (astrocytoma IDHmut/G4).

Embodiment 22: The method of any of embodiments 15-21, wherein the subject has methylguanine-methyltransferase-promoter methylation.

Embodiment 23: The method of any of embodiments 15-22, wherein after treatment the subject has ≥50% compliance with applying the alternating electric field.

Embodiment 24: The method of any of embodiments 15-23, wherein after treatment the subject has an increased survival probability compared to subjects having wild type IDH.

Embodiment 25: The method of any of embodiments 15-24, wherein after treatment the subject has an increased overall survival compared to subjects having wild type IDH.

Embodiment 26: The method of any of embodiments 15-25, wherein after treatment the subject has an increased overall survival when the subject has ≥50% compliance with applying the alternating electric field compared to a subjecting that has ≤50% compliance.

Embodiment 27: The method of any of embodiments 15-26, further comprising administering radiation therapy to the subject.

Embodiment 28: The method of embodiment 27, wherein the radiation therapy is administered before, during, and/or after applying the alternating electric field.

Embodiment 29: The method of embodiment 27 or 28, wherein the radiation therapy is administered before, during, and/or after administering the chemotherapy.

Embodiment 30: A method of treating a subject comprising: identifying a subject with methylguanine-methyltransferase-promoter (MGMT) methylation; and applying an alternating electric field to a target site of the subject for a period of time, wherein the target site comprises one or more cancer cells.

Embodiment 31: The method of embodiment 30, wherein identifying a subject with MGMT methylation comprises sequencing.

Embodiment 32: The method of any one of embodiments 30-31, further comprising identifying a subject with an IDHmut

Embodiment 33: The method of embodiment 32, wherein identifying a subject with an IDHmut comprises immunohistochemistry and/or sequencing.

Embodiment 34: The method of any one of embodiments 30-33, further comprising administering a therapeutically effective amount of chemotherapy to the subject.

Embodiment 35: The method of embodiment 34, wherein the chemotherapy is temozolomide (TMZ).

Embodiment 36: The method of any one of embodiments 34-35, wherein the chemotherapy is administered before, during, and/or after applying the alternating electric field.

Embodiment 37: The method of any of embodiments 30-36, wherein the subject has brain cancer.

Embodiment 38: The method of any of embodiments 30-37, wherein the subject has IDHmut WHO grade 4 astrocytoma (astrocytoma IDHmut/G4).

Embodiment 39: The method of any of embodiments 30-38, wherein the subject has methylguanine-methyltransferase-promoter methylation.

Embodiment 40: The method of any of embodiments 30-39, wherein after treatment the subject has ≥50% compliance with applying the alternating electric field.

Embodiment 41: The method of any of embodiments 30-40, wherein after treatment the subject has an increased survival probability compared to subjects having wild type IDH.

Embodiment 42: The method of any of embodiments 30-41, wherein after treatment the subject has an increased overall survival compared to subjects having wild type IDH.

Embodiment 43: The method of any of embodiments 30-42, wherein after treatment the subject has an increased overall survival when the subject has ≥50% compliance with applying the alternating electric field compared to a subjecting that has ≤50% compliance.

Embodiment 44: The method of any of embodiments 30-43, further comprising administering radiation therapy to the subject.

Embodiment 45: The method of embodiment 44, wherein the radiation therapy is administered before, during, and/or after applying the alternating electric field.

Embodiment 46: The method of embodiment 44 or 45, wherein the radiation therapy is administered before, during, and/or after administering the chemotherapy.

Embodiment 47: The method of any of the preceding embodiments, wherein the alternating electric field has a frequency between 50 kHz and 1 MHz.

Embodiment 48: The method of any of the preceding embdiment s, wherein the alternating electric field has a frequency of 150 kHz or 200 kHz.

Embodiment 49: The method of any of the preceding embodiments, wherein the alternating electric field has a field strength of between 0.5 and 4 V/cm RMS.

Embodiment 50: The method of any of the preceding embodiments, wherein the alternating electric field has a field strength of about 0.9 V/cm RMS.

Embodiment 51: The method of any of the preceding embodiments, wherein the subject has not previously been treated with an alternating electrical field.

Embodiment 52: The method of any of the preceding embodiments, wherein the subject has not previously been treated with any type of electrical field.

Embodiment 53: The method of any of the preceding embodiments, wherein the subject has undergone surgery.

Embodiment 54: The method of any of the preceding embodiments, wherein the subject has undergone a biopsy.

Embodiment 55: The method of any of the preceding embodiments, wherein the subject has not received prior chemotherapy for a brain tumor.

Embodiment 56: The method of any of the preceding embodiments, wherein the subject has not received prior radiation therapy to the brain.

Embodiment 57: The method of any of the preceding embodiments, wherein the subject has not previously treated with TMZ.

Embodiment 58: An alternating electric field for use in a method of treating of a subject having cancer comprising: wherein a target site receiving the alternating electric field comprises one or more cancer cells, wherein the subject has an isocitrate dehydrogenase mutation (IDHmut).

Embodiment 59: Embodiment 58, further comprising administering a therapeutically effective amount of chemotherapy to the subject.

Embodiment 60: Embodiment 59, wherein the chemotherapy is temozolomide (TMZ).

Embodiment 61: Embodiments 59-60, wherein the chemotherapy is administered before, during, and/or after applying the alternating electric field.

Embodiment 62: Any of the preceding embodiments, wherein the subject has brain cancer.

Embodiment 63: Any of the preceding embodiments, wherein the subject has IDHmut WHO grade 4 astrocytoma (astrocytoma IDHmut/G4).

Embodiment 64: Any of the preceding embodiments, wherein the subject has methylguanine-methyltransferase-promoter methylation.

Embodiment 65: Any of the preceding embodiments, wherein the subject has ≥50% compliance with applying the alternating electric field.

Embodiment 66: Any of the preceding embodiments, wherein after treatment the subject has an increased survival probability compared to subjects having wild type IDH.

Embodiment 67: Any of the preceding embodiments, wherein after treatment the subject has an increased overall survival compared to subjects having wild type IDH.

Embodiment 68: Any of the preceding embodiments, wherein after treatment the subject has an increased overall survival when the subject has ≥50% compliance with applying the alternating electric field compared to a subjecting that has ≤50% compliance.

Embodiment 69: Any of the preceding embodiments, further comprising administering radiation therapy to the subject.

Embodiment 70: Embodiment 69, wherein the radiation therapy is administered before, during, and/or after applying the alternating electric field.

Embodiment 71: Embodiment 69 or 70, wherein the radiation therapy is administered before, during, and/or after administering the chemotherapy.

Embodiment 72: An alternating electric field for use in a method of treating a subject comprising: identifying a subject with an isocitrate dehydrogenase mutation (IDHmut); wherein a target site receiving the alternating electric field comprises one or more cancer cells.

Embodiment 73: Embodiment 72, wherein identifying a subject with an IDHmut comprises immunohistochemistry and/or sequencing.

Embodiment 74: Embodiments 72-73, further comprising administering a therapeutically effective amount of chemotherapy to the subject.

Embodiment 75: Embodiment 74, wherein the chemotherapy is temozolomide (TMZ).

Embodiment 76: Any one of embodiments 74-75, wherein the chemotherapy is administered before, during, and/or after applying the alternating electric field.

Embodiment 77: Any of embodiments 72-76, wherein the subject has brain cancer.

Embodiment 78: Any of embodiments 72-77, wherein the subject has IDHmut WHO grade 4 astrocytoma (astrocytoma IDHmut/G4).

Embodiment 79: Any of embodiments 72-78, wherein the subject has methylguanine-methyltransferase-promoter methylation.

Embodiment 80: Any of embodiments 72-79, wherein after treatment the subject has ≥50% compliance with applying the alternating electric field.

Embodiment 81: Any of embodiments 72-80, wherein after treatment the subject has an increased survival probability compared to subjects having wild type IDH.

Embodiment 82: Any of embodiments 72-81, wherein after treatment the subject has an increased overall survival compared to subjects having wild type IDH.

Embodiment 83: Any of embodiments 72-82, wherein after treatment the subject has an increased overall survival when the subject has ≥50% compliance with applying the alternating electric field compared to a subjecting that has ≤50% compliance.

Embodiment 84: The method of any of embodiments 72-83, further comprising administering radiation therapy to the subject.

Embodiment 85: The method of embodiment 84, wherein the radiation therapy is administered before, during, and/or after applying the alternating electric field.

Embodiment 86: The method of embodiment 84 or 85, wherein the radiation therapy is administered before, during, and/or after administering the chemotherapy.

Embodiment 87: An alternating electric field for use in a method of treating a subject comprising: identifying a subject with methylguanine-methyltransferase-promoter (MGMT) methylation, wherein a target site receiving the alternating electric field comprises one or more cancer cells.

Embodiment 88: Embodiment 87, wherein identifying a subject with MGMT methylation comprises sequencing.

Embodiment 89: Any one of embodiments 87-88, further comprising identifying a subject with an IDHmut.

Embodiment 90: The method of embodiment 89, wherein identifying a subject with an IDHmut comprises immunohistochemistry and/or sequencing.

Embodiment 91: Any one of embodiments 87-90, further comprising administering a therapeutically effective amount of chemotherapy to the subject.

Embodiment 92: Embodiment 91, wherein the chemotherapy is temozolomide (TMZ).

Embodiment 93: Any one of embodiments 91-92, wherein the chemotherapy is administered before, during, and/or after applying the alternating electric field.

Embodiment 94: Any of embodiments 87-93, wherein the subject has brain cancer.

Embodiment 95: Any of embodiments 87-94, wherein the subject has IDHmut WHO grade 4 astrocytoma (astrocytoma IDHmut/G4).

Embodiment 96: Any of embodiments 87-95, wherein the subject has methylguanine-methyltransferase-promoter methylation.

Embodiment 97: Any of embodiments 87-96, wherein after treatment the subject has ≥50% compliance with applying the alternating electric field.

Embodiment 98: Any of embodiments 87-97, wherein after treatment the subject has an increased survival probability compared to subjects having wild type IDH.

Embodiment 99: Any of embodiments 87-98, wherein after treatment the subject has an increased overall survival compared to subjects having wild type IDH.

Embodiment 100: Any of embodiments 87-99, wherein after treatment the subject has an increased overall survival when the subject has ≥50% compliance with applying the alternating electric field compared to a subjecting that has ≤50% compliance.

Embodiment 101: Any of embodiments 87-100, further comprising administering radiation therapy to the subject.

Embodiment 102: Embodiment 101, wherein the radiation therapy is administered before, during, and/or after applying the alternating electric field.

Embodiment 103: Embodiment 101 or 102, wherein the radiation therapy is administered before, during, and/or after administering the chemotherapy.

Embodiment 104: Any of the preceding embodiments, wherein the alternating electric field has a frequency between 50 kHz and 1 MHz.

Embodiment 105: Any of the preceding embodiments, wherein the alternating electric field has a frequency of 150 kHz or 200 kHz.

Embodiment 106: Any of the preceding embodiments, wherein the alternating electric field has a field strength of between 0.5 and 4 V/cm RMS.

Embodiment 107: Any of the preceding embodiments, wherein the alternating electric field has a field strength of about 0.9 V/cm RMS.

Embodiment 108: Any of the preceding embodiments, wherein the subject has not previously been treated with an alternating electrical field.

Embodiment 109: Any of the preceding embodiments, wherein the subject has not previously been treated with any type of electrical field.

Embodiment 110: Any of the preceding embodiments, wherein the subject has undergone surgery.

Embodiment 111: Any of the preceding embodiments, wherein the subject has undergone a biopsy.

Embodiment 112: Any of the preceding embodiments, wherein the subject has not received prior chemotherapy for a brain tumor.

Embodiment 113: Any of the preceding embodiments, wherein the subject has not received prior radiation therapy to the brain.

Embodiment 114: Any of the preceding embodiments, wherein the subject has not previously treated with TMZ.

Embodiment 115: Chemotherapy, or a chemotherapeutic agent, for use in the method of any of Embodiments 1 to 57, wherein the method comprises administering a therapeutically effective amount of the chemotherapy or chemotherapeutic agent lto the subject.

Embodiment 116: Radiation therapy, or a radiation therapy agent, for use in the method of any of Embodiments 1 to 57, wherein the method comprises administering a therapeutically effective amount of the radiation therapy or radiation therapy agent to the subject.

1. A method of treating of a subject having cancer comprising:
applying an alternating electric field to a target site of the subject for a period of time, wherein the target site comprises one or more cancer cells, wherein the subject has an isocitrate dehydrogenase mutation (IDHmut).

2. A method of treating a subject comprising:
a. identifying a subject with an isocitrate dehydrogenase mutation (IDHmut); and
b. applying an alternating electric field to a target site of the subject for a period of time, wherein the target site comprises one or more cancer cells.

3. The method of claim 2, wherein identifying a subject with an IDHmut comprises immunohistochemistry and/or sequencing.

4. The method of claim 2, further comprising administering a therapeutically effective amount of chemotherapy to the subject.

5. The method of claim 4, wherein the chemotherapy is temozolomide (TMZ).

6. The method of claim 4, wherein the chemotherapy is administered before, during, and/or after applying the alternating electric field.

7. The method of claim 2, wherein the subject has brain cancer.

8. The method of claim 2, wherein the subject has IDHmut WHO grade 4 astrocytoma (astrocytoma IDHmut/G4).

9. The method of claim 2, wherein the subject has methylguanine-methyltransferase-promoter methylation.

10. The method of claim 2, wherein after treatment the subject has ≥50% compliance with applying the alternating electric field.

11. The method of claim 2, wherein after treatment the subject has an increased survival probability and/or overall survival compared to subjects having wild type IDH.

12. The method of claim 2, wherein after treatment the subject has an increased overall survival when the subject has ≥50% compliance with applying the alternating electric field compared to a subjecting that has ≤50% compliance.

13. The method of claim 2, further comprising administering radiation therapy to the subject.

14. The method of claim 13, wherein the radiation therapy is administered before, during, and/or after applying the alternating electric field.

15. The method of claim 13, wherein the radiation therapy is administered before, during, and/or after administering the chemotherapy.

16. The method of claim 2, wherein the alternating electric field has a frequency between 50 kHz and 1 MHz.

17. The method of claim 2, wherein the alternating electric field has a frequency of 150 kHz or 200 kHz.

18. The method of claim 2, wherein the alternating electric field has a field strength of between 0.5 and 4 V/cm RMS.

19. The method of claim 2, wherein the subject has not previously been treated with an alternating electrical field or has not previously been treated with any type of electrical field.

20. The method of claim 2, wherein the subject has undergone surgery, has undergone a biopsy, has not received prior chemotherapy for a brain tumor, has not received prior radiation therapy to the brain, and/or has not previously treated with TMZ.
